(19) United States
(12) Patent Application Publication
(10) Pub. No.: US 2016/0243238 A1
(43) Pub. Date: Aug. 25, 2016

(54) Compositions for Dermatological Use

(71) Applicant: CIPHER PHARMACEUTICALS, Mississauga (CA)

(72) Inventor: Morten SLOTH WEIDNER, Virum (DK)

(21) Appl. No.: 15/027,831

(22) PCT Filed: Oct. 28, 2014
(86) PCT No.: PCT/DK2014/050354

§ 371 Date: Apr. 7, 2016

(30) Foreign Application Priority Data
Oct. 28, 2013 (DK) PA 2013 00614

(51) Int. Cl. A61K 47/06; A61K 47/14; A61K 47/20

(52) U.S. Cl. CPC A61K 47/06; A61K 47/20; A61K 47/14

(57) ABSTRACT

The invention concerns novel compositions, and their use in the management of conditions associated with a defective or disrupted skin barrier in humans and animals. In particular, the invention relates to a colloid system resembling a cream with improved barrier properties. The invention may inter alia be used to diminish transepidermal water loss.

The present invention relates to novel compositions and their use in the management of conditions associated with a defective or disrupted skin barrier in humans and animals. In particular, the invention relates to a colloid system resembling a cream with improved barrier properties. The invention may inter alia be used to diminish transepidermal water loss.

### TECHNICAL BACKGROUND

The skin is the largest organ of the human body, covering an area of approximately 2 m². It is the interface with the external environment, and prevents the penetration of foreign molecules and the loss of water and endogenous substances. It is essentially composed of two major layers: the epidermis, an unvascularized layer, and the underlying dermis which contains a rich supply of capillaries, nerves, sweat and sebaceous glands and hair follicles, supported by connective tissue. The barrier function of the skin resides primarily in the epidermis and is localized in it's outer layer the stratum corneum, which is typically 10 to 20 μm thick.

The process of terminal differentiation of the epidermis (keratinization) leads to formation of the stratum corneum, which is an anucleate layer. Filament aggregating protein (filaggrin) is a key protein that binds to and is responsible for the aggregation of keratins (K1/10) which induce the cytoskeleton to collapse and result in formation of corneocytes.

The stratum corneum is a multilayered tissue composed of flattened, anucleate corneocytes, surrounded by multiple planar lamellae sheets, enriched in ceramides, cholesterol, and free fatty acids. The localization of these highly hydrophobic lipids within the extracellular domains of the stratum corneum inhibits the outward movement of water.

These lipids are delivered to the stratum corneum as their precursors through secretion of a unique organelle, the epidermal lamellar body. As the stratum corneum forms, this organelle delivers lipid constituents (cholesterol), lipid precursors (glucosylceramides and phospholipids), and enzymes (β-glucocerebrosidase, acidic sphingomyelinase, and secretory phospholipase A2) required to generate ceramides and free fatty acids, which are needed for their organization into mature membrane structures. In parallel, lamellar body-derived proteases and their inhibitors orchestrate the orderly digestion of corneodesmosomes, transient intercellular junctions that are progressively degraded, allowing corneocytes to shed invisibly at the skin surface. A fine balance between basal cell proliferation and corneocyte desquamation maintains the skin barrier at a constant thickness.

Due to the physiological significance of the skin barrier the abovementioned processes in the stratum corneum are under tight biochemical regulation. Even though this regulation is far from fully understood a number of mediators secreted by basal keratinocytes can up and down regulate ceramide synthesis, filaggrin expression etc. Thus mediators usually attributed to the immune system can be secreted by keratinocytes and affect the skin barrier through modulation of the stratum corneum.

The unique structure of the stratum corneum generates protective and defensive functions of essential importance to the organism.

Of these functions, the permeability barrier is probably the most critical. Thus it retards transcutaneous evaporative water loss, allowing survival in a potentially desiccating external environment.

Another important function of the stratum corneum is the prevention of foreign matter or microbes entering the body. In addition to the barrier preventing microbial invasion, antimicrobial peptides are delivered to the stratum corneum intercellular domains via secretion of lamellar body contents of such peptides.

Inter- and intra-variability in human skin barrier as a function of anatomic site, age, gender and ethnic differences have been reported and reviewed by a number of authors. The clinical methods typically used to assess such variation include trans-epidermal water loss (TEWL) measurement, epidermal hydration level (Corneometer) measurement and spectroscopic skin analysis. Epidermal Corneometer skin hydration measurement is a widely used in conjunction with TEWL measurement in the determination of skin barrier function.

An inadequately functioning skin barrier may give rise to various discomforts of which dry skin is obvious and may be experienced as highly unpleasant.

Many factors are known to exacerbate dry skin, such as excessive use of detergents, cold weather, exposure to UV-light, etc. Such skin barrier dysfunction resulting in dry skin discomfort is experienced by a large proportion of the population on a regular basis.

Some professions are particularly exposed, such as metal workers or health care professionals washing hands with detergents many times a day.

Furthermore there is a substantial amount of data demonstrating that various skin disorders are associated with disturbances of skin barrier function as evidenced by an increase in transepidermal water loss (TEWL) and a decrease in water-binding properties.

Such dysfunctions of the skin barrier have been observed in relation to various inflammatory conditions of the skin, such as atopic dermatitis, contact dermatitis and psoriasis.

It even appears that the skin barrier function is altered in uninvolved skin of such patients.

Skin barrier dysfunction is generally treated with moisturising emollient creams. Such products serve the purpose of creating a synthetic skin barrier substitution on the external side of the stratum corneum by topical administration of emulsions with a high proportion of lipids. This type of products may give a temporary symptomatic relief but the effect is limited due to a fast turnover of such compositions on the skin surface.

There is a strong need for an effective modality for enhancing the skin's barrier function.

Alkyl sulfates are surface active agents that have been used widely in toothpastes, soaps and as emulsifiers in oil-in-water emulsions. Numerous compositions have been published using alkyl sulfates, especially sodium cetostearylsulfate, where oil-in-water emulsions are formed in compositions comprising a lipid an aqueous phase due to the high negative zeta potential induced by the alkyl sulfates upon ionization.

### SUMMARY OF THE INVENTION

The present invention relates to the surprising discovery that a composition comprising an aqueous phase, a lipid phase and alkylsulfates can surprisingly form a continuos or homogenous lipid phase upon the further addition of the short chain monoglyceride glyceryl monocaprylate to the composition. This is highly unexpected because the alkylsulfates due to their negative ionization result in highly negative Zeta potentials and lead to formation of oil-in-water emulsions. The monoglyceride glyceryl monocaprylate, which is used widely as co-emulsifier, surprisingly disrupts the oil-in-water structure formed by alkylsulfates leading to continuos lipid phase, which includes the water in an intra-lipid manner (as aqueous inclusions within the continuous lipid phase) and/or in an interstitial manner (as aqueous deposits within the lamellar folds of the continuous lipid phase).

As demonstrated in the examples a continuous phase with a high physical integrity can be obtained by including a high proportion of high melting lipids defined as lipids with a melting point above 35° C., e.g. long chain fatty alcohols, long chain alkanes, long chain triglycerides, waxes, etc.

Such compositions have never been described before and holds the advantage of forming a continuos lipid barrier, upon application to the skin, having a negative surface charge. The stratum corneum (outer layer of the skin) and it's lipid barrier are also negatively charged. The formulation of the invention is therefore electrostatically repelled and therefore forms a longer lasting physical barrier in the outer layer of the stratum corneum.

The inventors have found that the compositions of the invention can be used to counteract dysfunctions in the skin barrier function.

Thus, the inventors have found that the compositions of the invention provide a novel general principle for improving skin barrier function for example in relation to cosmetic conditions, such as dry skin, sunburn and signs of ageing. Furthermore, the compositions of the invention constitute a novel, effective and safe treatment for the repair of a damaged skin barrier in skin diseases like the variuous forms of dermatitis, pruritus, psoriasis, or other skin diseases associated with a malfunctioning skin barrier.

Certain aspects and embodiments of the present invention are provided in the claims. Additional aspects and embodiments are described herein. Features of the aspects and embodiments may be combined.

According to different aspects, the invention concerns the subject-matter of claims 1, 2, 13, 33, 34, 42, 43, 44, 45 and 46.

According to one aspect, the invention provides new therapeutic as well as non-therapeutic uses of the compositions of the invention in relation to skin barrier integrity and functionality related to human and animal health care.

Furthermore, as demonstrated in the examples, the compositions of the invention exert barrier repairing effects by counteracting dysfunctions in the stratum corneum and in mucous membranes. Since the skin structure of mammals is somewhat comparable to humans and the issues of barrier repair are equally relevant to mammals, the principle of the invention is relevant to various mammals besides humans. Therefore, the invention also provides new therapeutic uses of compositions of the invention in relation to improving or repairing the skin barrier or mucous membranes in humans and animals.

### DETAILED DISCLOSURE

The foregoing and other aspects of the present invention will now be described in more detail with respect to other embodiments described herein. It should be appreciated that the invention can be embodied in different forms and should not be construed as limited to the embodiments set forth herein. Further, additional embodiments according to the invention are mentioned in the claims.

Unless otherwise defined, all technical and scientific terms used herein have the same meaning as commonly understood by one of ordinary skill in the art to which this invention pertains. The terminology used in the description of the invention herein is for the purpose of describing particular embodiments only and is not intended to be limiting of the invention. As used in the description of the invention and the claims set forth herein, the singular forms “a”, “an” and “the” are intended to include the plural forms as well unless the text clearly indicates otherwise. The person skilled in the art understands that while the plural or singular form of nouns is used in certain places, the plural may cover the singular, and vice-versa. As used herein, the term “and/or” includes any and all combinations of one or more of the associated listed items.

The following phrases, terms and definitions are used herein:

The term “therapeutic use” refers to any application of the invention related to “treatment” as defined below.

The term “treat” and “treatment” refers to the application of the present invention resulting in a reduction of the severity of the subject's condition or a least the condition is partially improved or ameliorated and/or that so alleviation, mitigation or decrease at least one clinical symptom is achieved and/or there is a delay in the progression of the condition and/or prevention or delay of the onset of the condition. Thus, the term “treat” refers to both preventionally and therapeutic treatment regimes.

The term “reducing” or “reduce” refers to a decrease or diminishment in the specified activity of at least about 10%, 25%, 35%, 40%, 50%, 60%, 75%, 80%, 90%, 95% or more. In som embodiments, the reduction results in little or essentially no detectible activity (at most, an insignificant amount, e.g. less tha about 10% or even 5%).

The term “an effective amount” refers to an amount of the compounds or composition of the present invention that is sufficient to produce the desired effect. The effective amount will vary with the application for which the compound or composistion is being employed, the age and physical conditioin of the subject, the severity of the condition, the duration of the treatment, the nature of any concurrent treatment, the carrier used, and similar factors within the knowledge and expertise of those skilled in the art.

The term “Intralipid” or intralipid manner means aqueous inclusions within the continuous lipid phase. Lipids may be divided into eight categories: fatty acids, glycerolipids, glycerophospholipids, sphingolipids, saccharolipids, and polyketides (derived from condensation of ketoacyl subunits); and sterol lipids and prenol lipids (derived from condensation of isoprene subunits).

The term “in an interstitial manner” means aqueous deposits within the lamellar folds of the continuous lipid phase.

The present invention inter alia provides the compositions of the invention for use as a skin barrier improving agent or skin barrier repairing agent.

According to an embodiment, the invention concerns a composition comprising:
a) Water;
b) An emollient;
d) An anionic water soluble emulsifier; and
e) A lipid soluble emulsifier.

The composition is preferably a pharmaceutical composition. The composition is preferably a dermatological composition.

The emollient is preferably an oleaginous vehicle. The emollient preferably comprises or consists of liquid saturated hydrocarbons, more preferred light liquid paraffin.

Without being bound by theory, it is speculated that electrostatic repulsion means that a composition or cream of the invention is not absorbed quickly. Thus, an anionic emulsifier may ensure sufficient electrostatic repulsion to prevent quick absorption, e.g. by the skin.

The lipid soluble emulsifier preferably is an ingredient having HBL 5-6.

According to an embodiment, the invention concerns a composition comprising:
a) 15%-80% of an aqueous phase;
b) 15%-80% of a lipid phase;
d) 0.5%-2% of at least one unbranched alkylsulfate of C12-C20 or a salt thereof;
e) 0.2%-2.0% of glyceryl monocaprylate; and optionally
f) an antimicrobial preservative.

According to an embodiment, the invention concerns the composition, which is in the form of a continuous phase.

According to an embodiment, the invention concerns the composition, wherein the lipid phase comprises at least 5% high-melting lipids. The expression “high-melting lipids” refers to lipids having a melting point of above 37° C., such as preferably 38° C. or above, at 1 atm pressure. Having a lipid with a melting point above 37° C. carries the advantage that the lipid may cover the surface of the skin longer time than a lipid with a lower melting point.

According to an embodiment, the invention concerns the composition, wherein said at least one unbranched alkylsulfate of C12-C20 or salt thereof is sodium cetostearyl sulphate.

According to an embodiment, the invention concerns the composition, comprising:
a) Water;
b) An emollient;
c) A water soluble emulsifier;
d) An anionic water soluble emulsifier; and
e) A lipid soluble emulsifier.

According to an embodiment, the invention concerns the composition, comprising:
a) 15%-65% water;
b) 10%-60% of a branched or unbranched alkane of C12-C40;
c) 5%-20% of at least one branched or unbranched alkanol of C14-C30;
d) 0.5%-2% of at least one unbranched alkylsulfate of C12-C20 or a salt thereof;
e) 0.2%-2.0% of glyceryl monocaprylate; and optionally
f) an antimicrobial preservative.

The at least one branched or unbranched alkanol of C14-C30 may be measured by Gas chromatography-mass spectrometry (GC-MS).

According to an embodiment, the invention concerns the composition, wherein said branched or unbranched alkane of C12-C40 is selected among branched or unbranched alkane of C20-C40. According to an embodiment, the invention concerns the composition, wherein said branched or unbranched alkane of C12-C40 is paraffin wax.

According to an embodiment, the invention concerns the composition, wherein said branched or unbranched alkane of C12-C40 is light liquid paraffin. Suitable emollients may comprise, but are not limited to, lanolin, coconut oil, and mixtures of aforementioned.

According to an embodiment, the invention concerns the composition, wherein said at least one branched or unbranched alkanol of C14-C30 is selected among C15-C26, more preferred C16-C22, preferably C17-C19 alkanol.

According to an embodiment, the invention concerns the composition, wherein said at least one branched or unbranched alkanol of C14-C30 is cetostearyl alcohol. Suitable water soluble emulsifiers may comprise, but are not limited to, cetyl alcohol, stearyl alcohol, myristyl alcohol, and mixtures of aforementioned.

According to an embodiment, the invention concerns the composition, further comprising:
g) a humectant, preferably selected among glycerol and/or propylene glycol.

A humectant is a hygroscopic substance used to keep things moist. It is often a molecule with several hydrophilic groups, most often hydroxyl groups; however, amines and carboxyl groups, sometimes esterified, can be encountered as well (its affinity to form hydrogen bonds with molecules of water, is the crucial trait). A humectant attracts and retains the moisture in the air nearby via absorption, drawing the water vapor into and/or beneath the organism/object's surface. Humectants can be used in topical dosage forms to increase the solubility of a chemical compound's active ingredient(s), increasing the active ingredients' ability to penetrate skin, and/or its activity time.

Other suitable humectancs may comprise, but are not limited to, lactic acid, urea and/or sodium pyrrolidone carboxylate.

According to an embodiment, the invention concerns the composition, wherein the composition includes water in an intra-lipid and/or interstitial manner.

According to an embodiment, the invention concerns the composition, further comprising a bioactive ingredient.

According to an embodiment, the invention concerns a method, said method comprising:
i. Mixing:
a) Water;
b) An emollient;
c) A water soluble emulsifier;
d) An anionic water soluble emulsifier; and
e) A lipid soluble emulsifier;
thereby allowing micelles to form in the mixture; and
ii. Further allowing at least part of the micelles in the mixture to merge thereby obtaining homogenous particles, if necessary by heating said mixture to a temperature and a period of time sufficient to obtain homogenous particles.

The emollient acts as a barrier material for the skin. This barrier material is preferably not soluble or has very low solubility in water.

The term “emulsifier” and “surfactant” are used interchangeably here, without implying any specifying HLB. The term “HLB” is used as in “The HLB System—a time-saving guide to emulsifier selection”, ICI Americas Inc., Wilmington, Del.

The expression: “if necessary by heating said mixture to a temperature and a period of time sufficient to obtain homogenous particles” also covers the case wherein the mixture is already at a temperature after the Mixing of step i. to allow homogenous particles to be obtained. The temperature should preferably be above the melting point of lipid(s) of the mixture.

This method may be used for producing a composition according to the invention, which may be described as a colloid system. A colloid is a substance in which microscopically dispersed insoluble particles are suspended throughout another substance. The produced colloid system of the present invention may be allowed to cool before packaging or use. Surprisingly, the produced colloid system has proven stable after (optional) cooling.

A usual cream is an emulsion of oil and water. A cream is a topical preparation usually for application to the skin. Creams for application to mucous membranes such as those of the rectum or vagina are also used. An emulsion may comprise micelles, aggregates of surfactant molecules dispersed in the liquid. Micelles may appear as microscopically spherical entities.

While the term “emulsion” is used here, the present invention is not necessarily confined to mixtures of liquids. The term “micelles” is used here to refer to individual spherical entities in a mixture.

Surprisingly, a colloid system with cream-like properties, in terms of viscosity and applicability, has been discovered. The colloid system has improved barrier properties, and in a preferred embodiment a semi-translucent, glossy appearance.

Light liquid paraffin is a very highly refined mineral oil used in cosmetics and for medical purposes. It is known as “light mineral oil” in the US, and as “light liquid paraffin” outside of the US. It is inter alia described in “Handbook of Pharmaceutical Excipients”, Seventh Edition.

Light liquid paraffin is known to have a short-lived barrier effect upon topical application. However, this barrier effect quickly diminishes when applied to the skin as the light liquid paraffin diffuses into the skin. Surprisingly, this barrier effect may be sustained, when light liquid paraffin forms part of a colloid system according to the invention.

The present invention relates to a colloid system. In particular, it relates to a colloid system resembling a cream with improved barrier properties.

In particular, a colloid system of the invention may diminish or minimize transepidermal water loss (TEWL).

The colloid system of the invention may inter alia be used as a cream with attractive properties, comprising, but not limited to, that it may diminish TEWL; it may prevent or treat dry skin and/or eczema.

According to an embodiment, the invention concerns the method, wherein the heating is continued until all, or substantially all, micelles form part of homogenous particles.

Upon heating, the micelles of the system merge into homogenous particles, as observed by electron microscopy after drying, i.e. removal of water from the system.

According to an embodiment, the invention concerns the method, wherein the obtained colloid system provides a single solid phase microstructure as observed by electron microscopy after removal of water.

In the same way a solid solution alloy may provide a single solid phase microstructure, the mixture of the present invention may provide a single solid phase microstructure as observed by electron microscopy after removal of water.

According to an embodiment, the invention concerns the method, wherein the obtained colloid system comprises or consists of plates as observed by electron microscopy after removal of water.

According to an embodiment, the invention concerns the method, wherein the obtained colloid system is a one-phase system in the absence of water as observed by electron microscopy.

According to an embodiment, the invention concerns the method, wherein said emollient has a melting point below 37° C., more preferred below 20° C.

The emollient is preferably an oleaginous vehicle. The emollient is preferably liquid saturated hydrocarbons, more preferred light liquid paraffin.

According to an embodiment, the invention concerns the method, wherein said water soluble emulsifier consists of or comprises at least one branched or unbranched alkanol of C14-C30.

According to an embodiment, the invention concerns the method, wherein said water soluble emulsifier has a HBL of 10-20, more preferred about 15-16.

The water soluble emulsifier is preferably an o/w emulsifier. The water soluble emulsifier is preferably an alcohol.

According to an embodiment, the invention concerns the method, wherein said water soluble emulsifier is cetostearyl alcohol.

According to an embodiment, the invention concerns the method, wherein said anionic water soluble emulsifier comprises or consists of at least one unbranched alkylsulfate of C12-C20 or a salt thereof.

According to an embodiment, the invention concerns the method, wherein said anionic water soluble emulsifier is Sodium cetostearyl sulphate. Potential alternatives comprise, but are not limited to, a compound such as sodium lauryl sulphate.

The anionic water soluble emulsifier is preferably an anionic o/w (oil-in-water) emulsifier. The anionic water soluble emulsifier preferably has a HLB>10, more preferred >15, preferably >20, more preferred >25, preferably >30, more preferred >35. Sodium cetostearyl sulphate is also supplied under the trademark of LANETTE E.

According to an embodiment, the invention concerns the method, wherein said lipid soluble emulsifier has an HBL of 3-6, preferably 5-6.

The lipid soluble emulsifier is preferably a w/o (water-in-oil) emulsifier. The lipid soluble emulsifier may act as a preservative, having surfactant properties.

According to an embodiment, the invention concerns the method, wherein said lipid soluble emulsifier is a preservative.

According to an embodiment, the invention concerns the method, wherein said lipid soluble emulsifier is glycerol monocaprylate. Potential alternatives may comprise, but are not limited to, polyglyceryl 3-polyricinoleate, triglycerol diisostearate, polyglyceryl oleate, and lecithin.

According to an embodiment, the invention concerns the method, further comprising a preservative, such as a microbial preservative, which is benzyl alcohol. Potential alternative preservatives comprise, but are not limited to, sorbic acid.

According to an embodiment, the invention concerns the method, further comprising a humectant, which is glycerol.

According to an embodiment, the invention concerns the method, further comprising a barrier enhancer, preferably nicotinamide. Suitable barrier enhancers may comprise, but are not limited to, dexpanthenol and clocortolone (an active ingredient of CLODERM cream), as well as mixtures of aforementioned.

According to an embodiment, the invention concerns the method, wherein the amount of water is 30-70 weight %, preferably 40-60 weight % in the obtained colloid system.

According to an embodiment, the invention concerns the method, wherein the amount of light liquid paraffin is 10-30 weight %, preferably 15-25 weight %, more preferred about 20 weight % in the obtained colloid system.

According to an embodiment, the invention concerns the method, comprising an amount of glycerol of 1-10 weight %, preferably 3-8 weight %, more preferred about 5 weight % in the obtained colloid system.

According to an embodiment, the invention concerns a composition obtainable according to a method of the invention.

According to an embodiment, the invention concerns a composition comprising a colloid system, said system comprising a mixture of:
a) Water;
b) An emollient;
c) A water soluble emulsifier;
d) A anionic water soluble emulsifier; and
e) A lipid soluble emulsifier, preferably an ingredient having HBL 5-6;

Subject to the proviso that said mixture upon removal of water comprises homogenous particles and is substantially free of micelles.

According to an embodiment the invention concerns the composition, wherein micelles are not present.

According to an embodiment the invention concerns the composition, which is a cream.

According to an embodiment the invention concerns the composition for topical or dermatological use.

According to an embodiment the invention concerns the composition, further comprising an active pharmaceutical ingredient. While all known APIs are candidates for inclusion in the present composition, in particular APIs having a dermatological use are particularly relevant. Small molecules are particularly relevant, being easy to incorporate in the composition of the invention. Thus, APIs used in any existing cream are potential candidates for incorporation in a composition of the invention.

In particular, the present invention may be combined with one or more active pharmaceutical ingredients mentioned in the International patent application WO 2004 000333. The present invention is particularly suited for indications mentioned in the International patent application WO 2004 000333. These APIs include, but are not limited to, Niacinamide, N2-methyl-niacinamide, Aminoniacinamide, Thioniacinamide, and N2-ethyl-niacinamid.

Other APIs comprise steroids, such as topical corticosteroids, which may be included in a composition of the invention. Topical corticosteroids for eczema include hydrocortisone, which is the same as the naturally-occurring corticosteroid cortisol, and synthetic corticosteroids such as betametasone, fluticasone and mometasone, and derivatives, such as betamethasone dipropionate.

Skin disease is often accompanied by itching. Itching and mild pain can sometimes be controlled with soothing agents such as chamomile, eucalyptus, camphor, menthol, zinc oxide, talc, glycerin, and calamine, which may be included in a composition of the invention.

Antihistamines, which block certain types of allergic reactions, may be included in topical compositions to relieve the itching associated with allergic reactions. Doxepin is an effective topical antihistamine for many conditions. Other possible antihistmines include, but are not limited to, the group consisting of Brompheniramine, chlorpheniramine, debrompheniramine, dexchlorpheniramine, carbinoxamine, clemastine, diphenhydramine, pyrilamine, tripelennamine, tripolidine, methdilazine, bromodiphenhydramine, promethazine, azatadine, cyproheptadine, diphenylpyraline, doxylamine, trimeprazine, phenindamine, ketotifen, hydroxyzine, tazifylline, temelastine, meclizine, acrivastine, setastine, oxatomide, mequitazine, levocabastine, lodoxamide, rocastine, phenindamine, azelastine, and ebastine, fexofenadine, loratadine, descarboethoxy loratadine, astemizole, norastemizole, desmethylastemizole, cetirizine, acrivastine, and temelastine.

Additionally, or alternatively, pyridine compounds e.g. for the treatment of dermatitis, such as compounds mentioned in the European patent EP 1941881 B1, may be included in a composition of the invention.

According to an embodiment the invention concerns the composition, comprising an active pharmaceutical ingredient selected among the group consisting of acexamic acid, adapalene, apremilast, benzoyl peroxide, clocortolone pivalate, desonide, desoxymethasone, dexamethasone acetate, dexamethasone palmitate, dexamethasone sodium phosphate, dexamethasone-17,21-dipropionate, dexamethasone-17-valerate, dexamethasone-21-isonicotinate, diflorasone diacetate, diflucortolone, dimetindene, fluclorolone acetonide, fluocinolone acetonide, fluocinonide, fluocortolone, halobetasol, halobetasol propionate, iodine, p-amino benzoic acid, p-aminobenzoate potassium, pimecrolimus, potassium permanganate, povidone iodine, prednicarbate, triamcinolone acetonide-21-phosphate potassium, urea, metronidazole, sulfacetamide, azelaic acid, clindamycin, and erythromycin.

According to an embodiment the invention concerns the composition, for the prevention or treatment of a condition selected among dry skin and eczema.

According to an embodiment the invention concerns a use of the composition, for the prevention or treatment of a condition selected among dry skin and eczema.

According to an embodiment the invention concerns a use of the composition, for diminishing trans epidermal water loss.

According to an embodiment the invention concerns a method for the treatment or prevention in a subject suffering from of a dermatological condition associated with impaired skin barrier function, comprising administration of a composition according to the invention to said subject.

According to an embodiment the invention concerns a use of a composition according to the invention, for the treatment of a dermatological condition associated with impaired skin barrier function in a human or animal.

According to an embodiment the invention concerns a use of a composition according to the invention as a drug delivery system.

As appreciated by the person skilled in the art, the lipid phase may consist of a wide variety of different classes of lipids giving the formulation different organoleptic properties.

Optionally the compositions of the inventions may further contain humectants such as monosaccharides, oligosaccharides or glycerol.

Guidance for the preparation of compositions of the invention can be found in “Remington: The science and practice of pharmacy” 20th ed. Mack Publishing, Easton Pa., 2000 ISBN 0-912734-04-3 and “Encyclopaedia of Pharmaceutical Technology”, edited by Swarbrick, J. & J. C. Boylan, Marcel Dekker, Inc., New York, 1988 ISBN 0-8247-2800-9 or a newer edition. As well known to the skilled person, illustrative additives to topical compositions include, but is not limited to: ointment bases, solvents, buffering agents, pH-adjusting agents, preservatives, humectants, chelating agents, antioxidants, stabilizers, emulsifying agents, suspending agents, gel-forming agents, and skin protective agents.

In another embodiment the compositions of the invention have a lamellar structure.

In a further embodiment the compositions of the invention may further comprise a bioactive ingredient, e.g. a drug molecule or a cosmetically active agent.

### FIGURES

FIG. 1 shows an Optical Bright Field Microscopy image of the edge of a native sample of a composition of the invention at 20× objective. Bar=10 micrometers.

FIG. 2 shows an Optical Bright Field Microscopy image of A) the edge of a native composition of the invention, with approaching water-filled micropipette; B) the same edge of the native composition but now with the pipet expelling a small amount of water (water is drawn out by capillary forces) embedded in the composition; C) water has been expelled from the micropipette and the composition starts to spontaneously form large droplets at the water interface still maintaining the continuous nature of the composition of the invention. (20× objective. Bar=10 micrometers).

FIG. 3 shows a Scanning Electron Microscopy (SEM) image of a cryo-fractured sample of a composition of the invention at 1600× magnification.

FIG. 4 shows a Scanning Electron Microscopy (SEM) image of a cryo-fractured sample of a composition of the invention at 6000× magnification.

FIG. 5 shows a Scanning Electron Microscopy (SEM) image of a cryo-fractured sample of a composition of the invention at 1600× magnification.

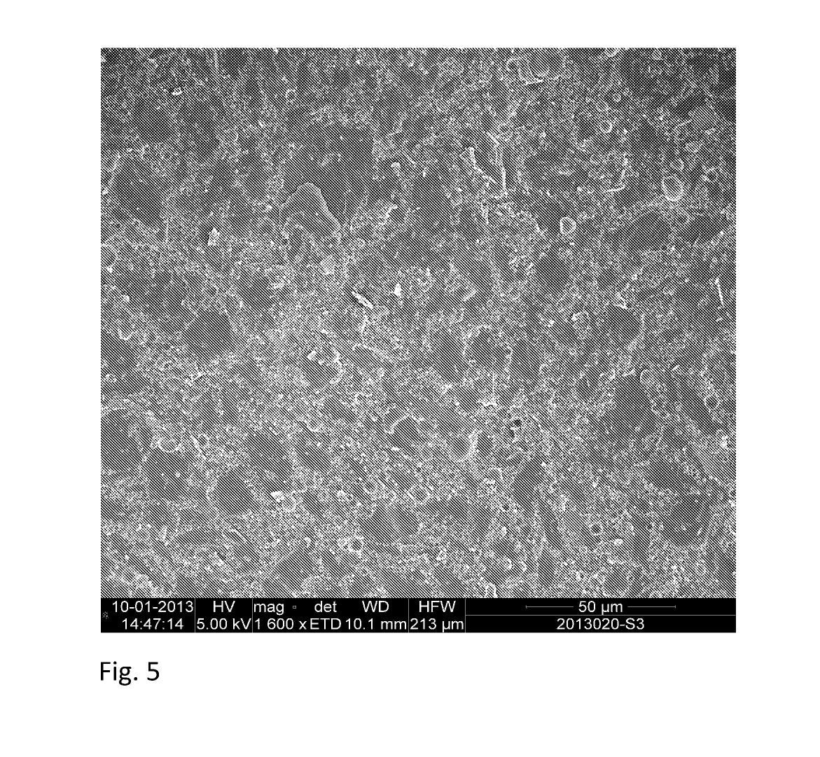

FIG. 6 shows a Scanning Electron Microscopy (SEM) image of a cryo-fractured sample of a composition of the invention at 6000× magnification.

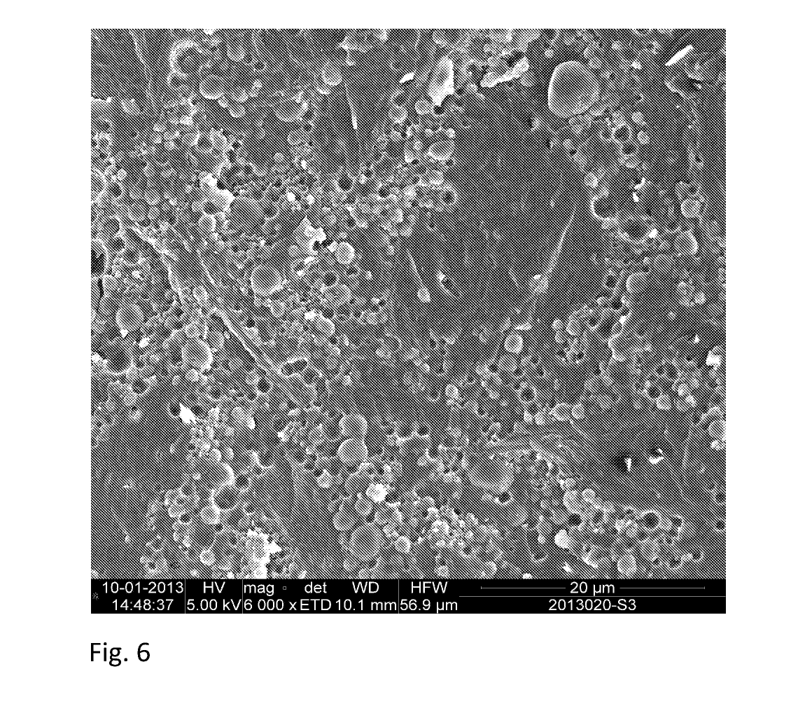

All cited references are incorporated by reference.

The accompanying Figures and Examples are provided to explain rather than limit the present invention. It will be clear to the person skilled in the art that aspects, embodiments and claims of the present invention may be combined.

Unless otherwise mentioned, all percentages are in weight/weight. Unless otherwise mentioned, all measurements are conducted under standard conditions (ambient temperature and pressure).

### EXAMPLES

### Example 1

This example concerns the preparation of a dermatological composition according to the invention comprising an emulsion containing alkylsulfate and glyceryl monocaprylate.

Objective

To prepare a composition according to the invention suitable for the uses and methods according to the invention.

Test Compounds and Chemicals

All chemicals employed were of standard analytical, cosmetic or pharmaceutical grade from diverse suppliers.

Test Formulations of the Invention

Test formulations, typically emulsions for topical use, according to the invention were prepared and one of them employed in the example below.

A general basic formulation was employed (“Standard Test Formulation” in the following), which could be varied for experimental purposes (all proportions weight/weight):

| | |
|---|---|
| Paraffin, Light Liquid | 20% |
| Cetostearyl alcohol | 10% |
| Glyceryl monocaprylate | 0.5% |
| Water, Purified | 63% |
| Glycerol 85% | 5% |
| Sodium cetostearyl sulphate | 1.0% |
| Benzyl Alcohol | 0.5% |

The method of preparing the formulation is as follows: A water phase (comprising water, glycerol, sodium cetostearyl sulphate, and benzyl alcohol) and a lipid phase (comprising paraffin and cetostearyl alcohol, but except glyceryl monocaprylate), are separately mixed and heated to 75° C., whereafter glyceryl monocaprylate is added to the lipid phase and dissolved. Then the two phases are mixed under homogenization until <30° C., providing the final composition ready for application.

### Example 2

This example is related to the biophysical characterization of the properties of the dermatological composition of the invention prepared in Example 1.

Microscopic Characterization of the Composition

Macroscopically the composition appears as a homogeneous white cream which is easily applied to the skin and dispersed on the skin surface.

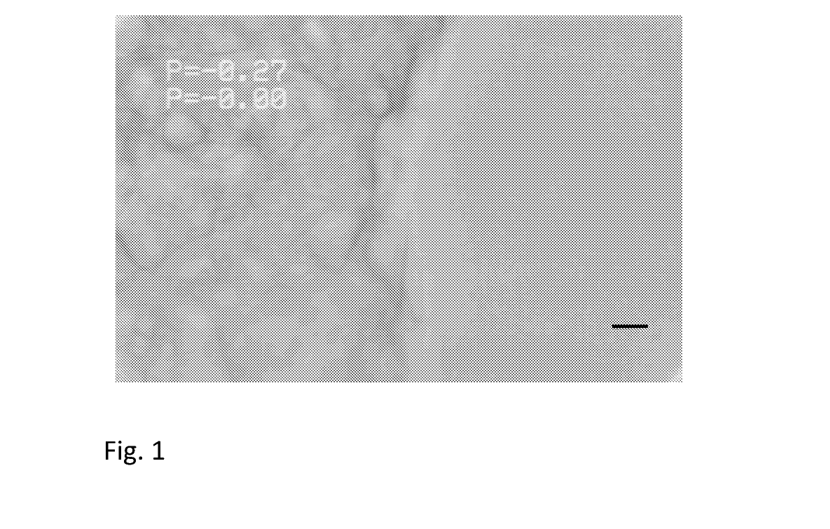

Optical bright field microscopy of a microscope slide preparation of the composition revealed a continuous phase with an irregular surface without emulsion particles (See FIG. 1).

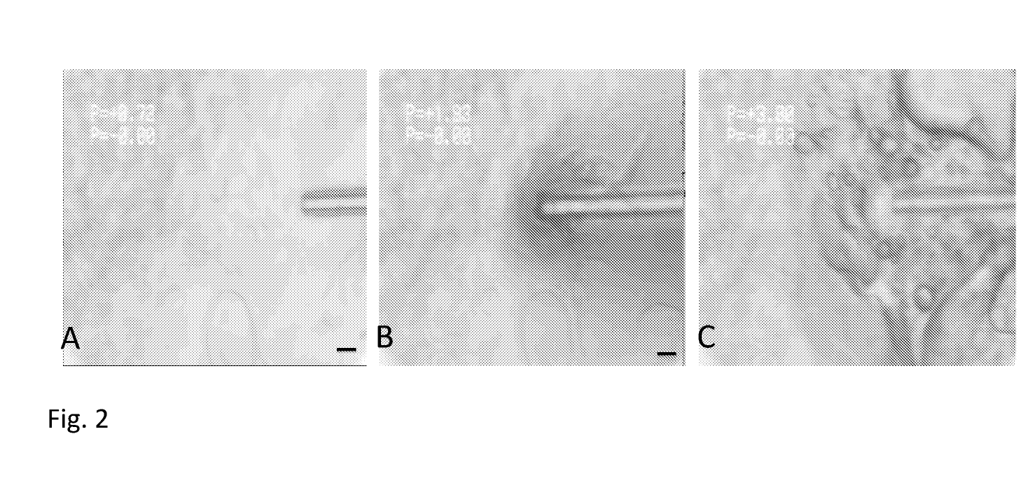

The continuous nature of the lipid phase was further confirmed by hydration of the composition under bright field microscopy with nano-liters of deionized water delivered by micropipette. This is shown in FIG. 2, where the addition of water leads to visible large droplets at the aqueous interface without breaking the continuous nature of the composition.

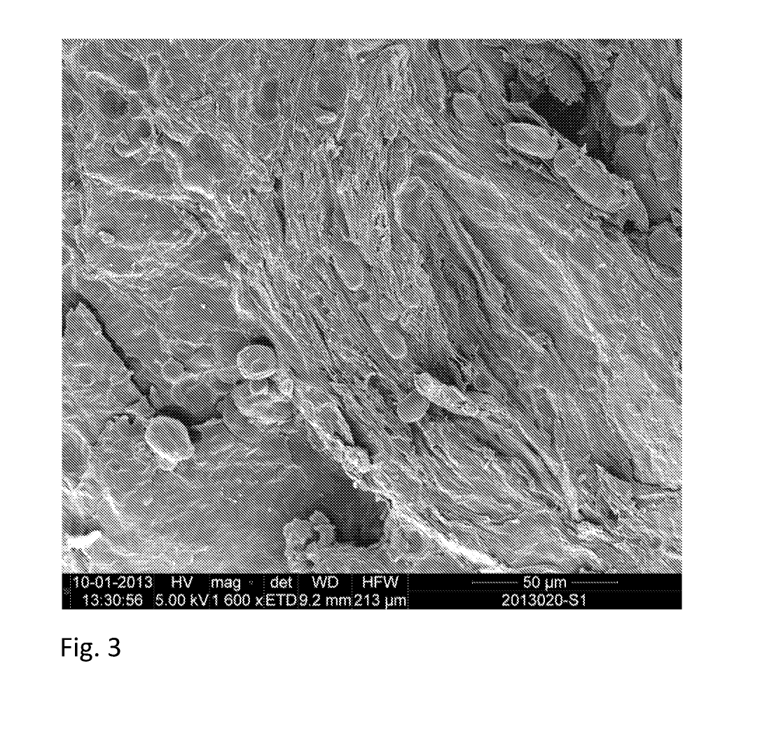

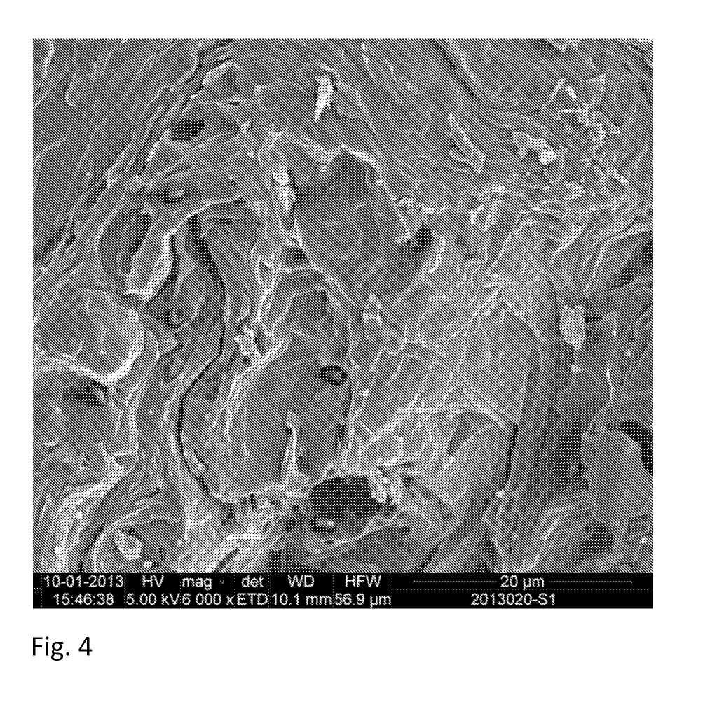

Scanning electron microscopy (SEM) of cryo-fixed and cryo-fractured samples of the composition of the invention are shown in FIG. 3 (1600× magnification) and FIG. 4 (6000× magnification). The samples are subjected to a sublimation step in which the surface water of the fracture is carefully removed without disturbing the general sample structure. Cryo-SEM is thus suitable for further evaluation of the organization of the lipid phase. The SEM images shown in FIGS. 3 and 4 clearly demonstrate the continuous nature of the lipid phase of the composition.

It is important to note that similar images of oil-in-water or water-in-oil emulsions reveal the characteristic homogenous signature of droplets of one phase dispersed in a continuous phase of the other. Such characteristics are not seen in the Cryo-SEM images of composition of the invention.

Furthermore, the Cryo-SEM images in FIGS. 3 and 4 demonstrate that the structural organization of the continuous lipid phase of the composition resembles a lamellar structure potentially capable of including water.

Further Structural and Phase Characterization of the Composition of the Invention

To test whether the continuous lamellar lipid phase of the composition includes water in an intra-lipid manner (as aqueous inclusions within the continuous lipid phase), in an interstitial manner (as aqueous deposits within the lamellar folds of the continuous lipid phase) or both, the composition was exposed to high-speed centrifugation to separate interstitial water from the composition. In this manner an interstitial water phase of 15.3% could be separated from the composition.

A comparable amount of interstitial water was observed by extracting a known quantity of the composition with weighed filter paper demonstrating an interstitial water phase of 17.5%.

Since the two methods of estimating the interstitial water phase confirmed a similar level, it was concluded that the interstitial water phase amounts to about 16% of the water in the composition.

On this basis about 84% of the water in Helioclin® Dermatitis SD Cream is concluded to be intra-lipid constituting aqueous inclusions in the continuous lipid phase of the composition.

These findings demonstrate that Helioclin® Dermatitis SD Cream is organized as a three compartment system.

To further characterize the phase characteristics of the dermatological composition of the invention, a phase transition analysis was performed by Differential Scanning calorimetry (DSC). This demonstrated a single exothermic peak during cooling of the composition (20° C.→−40° C.) at −11.5° C. related to transition of the aqueous phase from liquid to solid form. During the following heating of the composition (−40° C.→180° C.) the first endothermic peak occurred at 0.37° C. (onset at −6.46) related to the reverse transition of the solid aqueous phase to liquid state. This was followed by a less well defined endothermic peak with onset at approximately 50° C. and maximum at approximately 90° C. related to the transition of the lipid phase of the composition from solid to liquid state.

The DSC analysis demonstrated that the continuous lipid phase of the composition maintains its solid state and physical integrity at skin surface temperature.

Surface Charge Characterization of the Composition

In order to measure the surface charge characteristics of Helioclin® Dermatitis SD Cream, a 0.001% dispersion of the composition was prepared in KCl 1 mM by vortexing. The Zeta potential was measured employing a Zetasizer (Malvern Nano-ZS) and found to be −126 mV. This is in line with the presence of surface-active alkyl-sulphates in the composition giving (OSO3−) head-groups at the surface interface. The data show that the continuous lipid phase of the composition has a negative surface charge upon hydration.

Conclusion on Bio-Physical Characterization

The dermatological composition of the invention has the following properties according to the bio-physical characterization: The data indicate that the composition is a three compartment system comprising a lamellar continuous lipid phase with an intra-lipid aqueous phase and an interstitial aqueous phase. The continuous lipid phase is solid at skin surface temperature which ensures its physical integrity and durability as a barrier. The continuous and solid lipid phase ensures the formation of a durable continuous lipid barrier in the outer layer of the stratum corneum when the composition is applied to the skin giving optimal skin barrier properties.

The surface of the continuous lipid phase is negatively charged. This is an interesting finding, since both the epithelium and the stratum corneum barrier of the skin are also negatively charged, making an electrostatic repulsion of the composition possible, inhibiting the absorption of the composition and prolonging its barrier effect in the top layer of the stratum corneum.

The principal Mode of Action of the composition is likely physical/mechanical based on these bio-physical properties. The barrier protects against external irritants and reduces trans-epidermal water loss (TEWL), leading to increased stratum corneum hydration and improved symptoms of dermatitis that are well correlated with TEWL.

### Example 3

A clinical trial to treat facial eczema was carried out. The patients applied in average an amount of composition of the invention of 17.8 g per patient (with a standard deviation of 12.6 g). The patients treated the affected areas twice daily for three weeks. The treated area averaged 28.0 cm². The composition provided improvement of the condition exceeding the result expected from a placebo.

1. A composition comprising:
a) 15%-80% of an aqueous phase;
b) 15%-80% of a lipid phase;
d) 0.5%-2% of at least one unbranched alkylsulfate of C12-C20 or a salt thereof;
e) 0.2%-2.0% of glyceryl monocaprylate; and optionally
f) an antimicrobial preservative.

2. A composition comprising:
a) Water;
b) An emollient;
d) An anionic water soluble emulsifier; and
e) A lipid soluble emulsifier.

3. The composition according to claim 1 or 2, which is in the form of a continuous phase.

4. The composition according to any of the preceding claims, wherein the lipid phase comprises at least 5% high-melting lipids.

5. The composition according to any of the preceding claims, wherein said at least one unbranched alkylsulfate of C12-C20 or salt thereof is sodium cetostearyl sulphate.

6. The composition according to any of the preceding claims, comprising:
a) Water;
b) An emollient;
c) A water soluble emulsifier;
d) An anionic water soluble emulsifier; and
e) A lipid soluble emulsifier.

7. The composition according to any of the preceding claims, comprising:
a) 15%-65% water;
b) 10%-60% of a branched or unbranched alkane of C12-C40;
c) 5%-20% of at least one branched or unbranched alkanol of C14-C30;
d) 0.5%-2% of at least one unbranched alkylsulfate of C12-C20 or a salt thereof;
e) 0.2%-2.0% of glyceryl monocaprylate; and optionally
f) an antimicrobial preservative.

8. The composition according to claim 7, wherein said branched or unbranched alkane of C12-C40 is light liquid paraffin.

9. The composition according to claim 7 or 8, wherein said at least one branched or unbranched alkanol of C14-C30 is cetostearyl alcohol.

10. The composition according to any of the proceeding claims further comprising:
g) a humectant selected among glycerol and/or propylene glycol.

11. The composition according to any of the preceding claims, wherein the composition includes water in an intra-lipid and/or interstitial manner.

12. The composition according to any of the preceding claims, further comprising a bioactive ingredient.

13. A method for producing a composition, said method comprising:
i. Mixing:
a) Water;
b) An emollient;
c) A water soluble emulsifier;
d) An anionic water soluble emulsifier; and
e) A lipid soluble emulsifier;
thereby allowing micelles to form in the mixture; and
ii. Allowing at least part of the micelles in the mixture to merge, thereby obtaining homogenous particles, optionally by heating said mixture.

14. The method according to claim 13, wherein the heating is continued until all micelles form part of homogenous particles.

15. The method according to claim 13 or 14, wherein the obtained colloid system provides a single solid phase microstructure as observed by electron microscopy after removal of water.

16. The method according to any of the claims 13-15, wherein the obtained colloid system comprises or consists of plates as observed by electron microscopy after removal of water.

17. The method according to any of the claims 13-16, wherein the obtained colloid system is a one-phase system in the absence of water as observed by electron microscopy.

18. The method according to any of the claims 13-17, wherein said emollient has a melting point below 37° C., more preferred below 20° C.

19. The method according to any of the claims 13-18, wherein said water soluble emulsifier consists of or comprises at least one branched or unbranched alkanol of C14-C30.

20. The method according to any of the claims 13-19, wherein said water soluble emulsifier has a HBL of 10-20, more preferred about 15-16.

21. The method according to any of the claims 13-20, wherein said water soluble emulsifier is cetostearyl alcohol.

22. The method according to any of the claims 13-21, wherein said anionic water soluble emulsifier comprises or consists of at least one unbranched alkylsulfate of C12-C20 or a salt thereof

23. The method according to any of the claims 13-22, wherein said anionic water soluble emulsifier is Sodium cetostearyl sulphate.

24. The method according to any of the claims 13-23, wherein said lipid soluble emulsifier has an HBL of 3-6, preferably 5-6.

25. The method according to any of the claims 13-24, wherein said lipid soluble emulsifier is a preservative.

26. The method according to any of the claims 13-25, wherein said lipid soluble emulsifier is glycerol monocaprylate.

27. The method according to any of the claims 13-26, further comprising a preservative, which is benzyl alcohol.

28. The method according to any of the claims 13-27, further comprising a humectant, which is glycerol.

29. The method according to any of the claims 13-28, further comprising a barrier enhancer, preferably nicotinamide.

30. The method according to any of the claims 13-29, wherein the amount of water is 30-70 weight %, preferably 40-60 weight % in the obtained colloid system.

31. The method according to any of the claims 13-30, wherein the amount of light liquid paraffin is 10-30 weight %, preferably 15-25 weight %, more preferred about 20 weight % in the obtained colloid system.

32. The method according to any of the claims 13-31, comprising an amount of glycerol of 1-10 weight %, preferably 3-8 weight %, more preferred about 5 weight % in the obtained colloid system.

33. A composition obtainable according to any of the claims 13 to 32.

34. A composition comprising a colloid system, said system comprising a mixture of:
a) Water;
b) An emollient;
c) A water soluble emulsifier;
d) A anionic water soluble emulsifier; and
e) A lipid soluble emulsifier, preferably an ingredient having HBL 5-6;
Subject to the proviso that said mixture upon removal of water comprises homogenous particles and is substantially free of micelles.

35. The composition according to claim 33 or 34, wherein micelles are not present.

36. The composition according to any of the claims 33-35, as a cream.

37. The composition according to any of the claims 33-36, for topical or dermatological use.

38. The composition according to any of the claims 33-37, further comprising an active pharmaceutical ingredient.

39. The composition according to claim 38, wherein said active pharmaceutical ingredient is selected among corticosteroids and antihistamines.

40. The composition according to claim 38 or 39, wherein said active pharmaceutical ingredient is selected among the group consisting of acexamic acid, adapalene, apremilast, benzoyl peroxide, clocortolone pivalate, desonide, desoxymethasone, dexamethasone acetate, dexamethasone palmitate, dexamethasone sodium phosphate, dexamethasone-17,21-dipropionate, dexamethasone-17-valerate, dexamethasone-21-isonicotinate, diflorasone diacetate, diflucortolone, dimetindene, fluclorolone acetonide, fluocinolone acetonide, fluocinonide, fluocortolone, halobetasol, halobetasol propionate, iodine, p-amino benzoic acid, p-aminobenzoate potassium, pimecrolimus, potassium permanganate, povidone iodine, prednicarbate, triamcinolone acetonide-21-phosphate potassium, urea, metronidazole, sulfacetamide, azelaic acid, clindamycin, and erythromycin.

41. The composition according to any of the claims 33-40, for the prevention or treatment of a condition selected among dry skin and eczema.

42. A use of a composition according to any of the claims 1-12 or 33-41, for the prevention or treatment of a condition selected among dry skin and eczema.

43. A use of a composition according to any of the claims 1-12 or 33-41, for diminishing transepidermal water loss.

44. A method for the treatment or prevention in a subject suffering from of a dermatological condition associated with impaired skin barrier function, comprising administration of a composition according to any of the claims 1-12 or 33-41 to said subject.

45. A use of a composition according to any of the claims 1-12 or 33-41, for the treatment of a dermatological condition associated with impaired skin barrier function in a human or animal.

46. A use of a composition according to any of the claims 1-12 or 33-41, as a drug delivery system.
